(19) United States
(12) Patent Application Publication
(10) Pub. No.: US 2025/0147016 A1
(43) Pub. Date: May 8, 2025

(54) CHIP-BASED OPTICAL DETECTION SYSTEM

(71) Applicant: Panacea Diagnostics Ltd, London (GB)

(72) Inventors: Marko Dorrestijn, Lucerne (CH); David R. Klug, London (GB); Shao-Ju Lu, London (GB); Callum Robertson Smith, Kongens Lyngby (DK); Stefan Leo Van Workum, London (GB); Vlad Turek, London (GB)

(73) Assignee: Panacea Diagnostics Ltd, London (GB)

(21) Appl. No.: 19/015,198

(22) Filed: Jan. 9, 2025

Related U.S. Application Data
(63) Continuation of application No. 17/435,376, filed on Aug. 31, 2021.

(30) Foreign Application Priority Data
Mar. 1, 2019 (GB) 1902810.9

(51) Int. Cl. G01N 33/543; B01L 3/00; G01N 21/64

(52) U.S. Cl. CPC G01N 33/54373; B01L 3/5023; B01L 3/502715; B01L 3/502723; B01L 3/50273; B01L 3/502753; B01L 3/502761; G01N 21/6428; B01L 2200/0631; B01L 2200/0636; B01L 2200/10; B01L 2200/142; B01L 2300/0627; B01L 2300/0654; B01L 2300/0816; B01L 2300/0819; B01L 2400/0436; B01L 2400/0677; B01L 2400/082; B01L 2400/086

(57) ABSTRACT

A chip-based optical detection system for detecting a target component in a liquid sample is provided. The system comprises a fluid pathway disposed in an interior volume of the chip; an inlet fluidically connecting an external surface of the chip to the fluid pathway; an optical element at least partially defining the fluid pathway; and a flow controller configured to control a flow of the liquid sample through the fluid pathway. One or more capture components are immobilised on the optical element, one or more detection reagents are provided in a proximity of the capture components, and the flow controller is configured to control the flow of the liquid sample along the fluid pathway such that the detection reagents are brought into contact with the capture components via diffusion.

### RELATED APPLICATIONS

This application is a continuation of U.S. patent application Ser. No. 17/435,376, filed on Aug. 31, 2021, which was a national stage filing under 35 U.S.C. 371 of International Patent Application Serial No. PCT/GB2020/050482, filed Feb. 28, 2020, which claimed priority to GB application no. GB1902810.9, filed Mar. 1, 2019. Each of these applications is herein incorporated by reference, in its entirety, for all purposes.

### FIELD OF THE INVENTION

The present disclosure relates to optical target detection, more specifically a chip-based optical detection system for detecting a target component in a liquid sample.

### BACKGROUND OF THE INVENTION

Assay devices can accommodate samples of varying sizes, from just a few nl right up to a few ml. Sometimes, the entire sample is processed, but often only a proportion of the sample is fully processed. The sample may be processed in its native state or it may be mixed with reagents (e.g. detection antibodies) before being transported through a flow channel (or porous medium such as nitrocellulose membrane) to a test site. Reagents stored on the chip are either in a separate chamber or pouch (in a buffer solution) or disposed in the flow channel in a dry format. The former requires a pump and a mixing stage, the latter requires dissolution of a dry deposit. Both of these existing technologies are technically challenging and can limit assay performance. Any channels and pumps used for introducing reagent can be costly in terms of design time, fabrication, real estate on the device and loss of reagent due to nonspecific binding to the channel walls. Furthermore, non-specific binding of analyte to channel walls leads to reduced sensitivity and adds potential variability.

EP2627987B1, for example, discloses a fluidic cartridge with passively driven fluid flow. The disclosed device and method solve problems associate with passive-flow fluidics by using a wicking pad and/or a tilting means to prevent channel draining and backflow. Therefore, there is a requirement and a need in the technical field to develop simple, lower-cost and reliable alternative assay device and methods e.g. a disposable single use consumer chip.

It is against this background that the present invention has arisen.

### SUMMARY OF THE INVENTION

According to the present invention there is provided an assay cartridge for detecting a target component in a liquid sample, the cartridge comprising: a sample collection unit configured to introduce the liquid sample into the cartridge; a fluid pathway commencing at its proximal end at the sample collection unit and extending distally through the cartridge including: one or more capture components immobilised within the fluid pathway; one or more detection reagents provided within the diffusion distance of the capture components.

By providing the detection reagents and capture components within the diffusion distance obviates the need for premixing. The liquid sample is introduced to the cartridge and flows along the fluid pathway at a first, relatively rapid, initial velocity. Once the liquid sample reaches the vicinity of the capture components and detection reagents, its velocity is drastically reduced so that diffusion predominates. The capture components and detection reagents are provided within the diffusion distance and therefore they can combine with the sample without any need for premixing.

Furthermore, the provision of the capture components and the detection reagents very close together helps with certainty about the timing of the assay because the sample fluid will come into contact with both the capture components and the detection reagents almost simultaneously.

Additionally, the signal in a diffusion dominated assay is independent of the speed and/or directionality of the initial fill of the device as the bulk flow is subsequently slowed or brought to a complete stop. In a bulk flow dominated regimen, the flow speed has a direct impact on the signal as does the directionality of the flow. The selection of a diffusion dominated regimen avoids this.

Furthermore, diffusion dominated assays have a smaller footprint than bulk flow dominated embodiments. This means that diffusion dominated configuration take up less real estate on the chip. For example, bulk flow dominated embodiments require space to mix the detection reagent into the liquid sample before reaching the capture components. Furthermore, corrugated or serpentine channels are favored in free flow or bulk flow embodiments because of the requirement of a controlled flow rate.

The diffusion dominated regimen will allow well-based embodiments with a single liquid handling step, i.e. the fill of the sample. Current well-based assays require multiple liquid handling steps, as for example in ELISA assays.

According to the present invention there may be provided an assay cartridge for detecting a target component in a liquid sample, the cartridge comprising: a sample collection unit configured to introduce the liquid sample into the cartridge; a fluid pathway commencing at its proximal end at the sample collection unit and extending distally through the cartridge including: one or more capture components immobilised within the fluid pathway; one or more detection reagents provided proximally of or level with the capture components, wherein the or each detection reagent is contained within a liquid droplet.

Provision of the detection reagents in the form of liquid droplet enables them to be combined with the sample such that they undergo bulk movement with the liquid sample. In this “free-flow” configuration, the droplets remain in liquid form and are not absorbed into a porous matrix. As the liquid sample moves over the detection reagents, they are dragged along with the bulk movement of the liquid sample and, because the flow of the liquid sample is substantially laminar, they create streaks of detection reagent. In addition, from liquid drops, reagent can be mixed/dissolved more quickly into solution than from dry deposits.

In some embodiments, the liquid sample can be permitted to absorb into a matrix provided on the fluid pathway or from which the fluid pathway is at least partially fabricated. The provision of an absorbent matrix into which the detection reagent can absorb provides an advantage in that it can allow a relatively large volume of reagent solution to be stored per unit length of the pathway. This is appropriate for the detection reagents because they can be provided in excess unlike the capture components which are provided as a monolayer where the number of capture components is precisely known and controlled.

The detection of the target component may include identifying the presence of the component. Detection may also include identifying the amount of at least one target component present in the liquid sample.

The fluid pathway may commence at a location at which the sample is introduced into the cartridge. The fluid pathway also includes the location of the capture component or components and any intervening geometry. The capture components may be located in a well. Alternatively, the fluid pathway may be elongate and comprise one or more elongate walls. The capture components may be located on one of more walls of the fluid pathway.

The fluid pathway may have a rectangular or square cross section comprising four substantially orthogonal walls. The walls are substantially perpendicular to provide a fluid pathway of substantially constant cross section. In these embodiments, the capture components may be located at the same distal distance along the fluid pathway as the detection reagents, but on different walls. The capture components may be supplied on orthogonally opposed walls to the detection reagents or they may be provided on adjacent walls.

The fluid pathway may be elongate and cylindrical and may include a single annular wall. In the embodiment having a cylindrical fluid pathway the capture components may be located at the same annular position on the wall, but different distal distances along the wall. Alternatively, the capture components may be located in different annular positions on the wall, at the same distance from the sample collection unit.

Configurations in which the detection reagents and capture components are provided at the same distal distance along the fluid pathway is more appropriate for a diffusion dominated flow regimen because the liquid sample will arrive in the vicinity of the detection reagents and capture components at approximately the same time. Once bulk flow has reached the detection reagents and capture components, the bulk flow can be slowed to a point where diffusion predominates or bulk flow can be stopped completely. By having the capture components and detection reagents separated in the annular direction, or by positioning them on adjacent or opposing walls, they cannot come into contact one with the other in the absence of the liquid sample. However, once the liquid sample is present, the capture components and detection reagents can move diffusively within the liquid sample and interact with the target component within the fluid sample.

The fluid pathway may be a well. The well configuration is particularly advantageous when the process for combining the liquid sample with the detection reagents and capture components is diffusion dominated. In such embodiments, the well wall can stop or considerably reduce the bulk movement of the liquid sample. The enables diffusional movement to predominate. The liquid sample will dissolve detection reagents that are present on the walls of the fluid pathway. The capture components that are located on an adjacent wall remain bound to the surface thereby providing localisation of the sandwich assay created by the combination of the detection reagent, target component and capture component when the target components encounter the capture components and detection reagents through diffusional movement within the well.

The detection reagents may be placed on different walls. The detection reagents may be provided on a wall which is, in use, illuminated by a light beam. Alternatively, the detection reagents may be provided on a wall that is not illuminated by a light beam.

Alternatively, the detection reagents may be substantially equidistant with the capture components. That is, the distance from the point at which the sample is introduced into the device is the same for the capture components and the detection reagents. This is in order to create maximum concentration of detection reagent for a certain disposed amount.

The detection reagents may be separated from the capture components by less than the diffusion distance achievable in the duration of the analysis.

Where the positioning of the detection reagents relative to the capture components is such that the target component and detection reagents will flow over the capture components then the diffusion length can be less than the spot diameter of the capture components.

The separation of the detection reagents from the capture components by less than the diffusion distance achievable in the duration of the analysis reduces overall background signal from unbound detection reagents, to avoid cross-talk (e.g. antibody-antibody cross-talk), and to reduce the required amount of costly reagent by reducing the liquid volume over which the reagent gets distributed.

Detection reagents and capture components are typically dispensed onto assays in the form of liquid droplets, using contact or non-contact printers or spotters. However, the water will have evaporated by the time the final product is ready for use (even when ignoring shelf life), unless evaporation is mitigated with appropriate measures. Keeping the droplets substantially liquid for the duration of the device's shelf life will facilitate their mixing with the sample, thus allowing the droplet contents to be readily transported by the sample flow and/or to readily diffuse through the sample. Retaining the water can also help maintain the conformations of solutes, e.g. preventing biomolecules from denaturing.

At least one of the liquid droplets may comprise an additive that minimises or even halts evaporation. This ensures that the liquid droplet is retained in liquid form and does not transition to a dried spot, either on a surface or within a matrix. The additive may be a hygroscopic compound such as a betaine, a sugar, a polyol, or an amino acid. In one embodiment, the betaine trimethylglycine is added in 1.4 M concentration. In another embodiment, sucrose is added at 0.88M (30% w/v). In another embodiment, the polysaccharide trehalose is added at 0.88M.

To further reduce or halt evaporation, the humidity in the fluid pathway can be elevated. One method to achieve this is by disposition of one or more droplets of salt solution inside the fluid pathway. The relative humidity can be controlled by the type and concentration of salt. To avoid condensation inside the device, the relative humidity must remain below 100% for the specified temperature range of the device. Using, for example, saturated solutions of ammonium sulphate, the relative humidity can be maintained in a narrow range of 79-82% for a temperature range of 0-50° C. To avoid interaction of the salt solution with the assay, these droplets are preferentially disposed downstream of the test site.

Another method for reducing evaporation is reduction of droplet's surface-to-volume ratio. This can be achieved by disposing at least one of the liquid droplets in one or more indentations, such as a recess, a trough, a ditch, a trench, a groove, a gully, a via that is essentially perpendicular to the surface, or in a porous structure. Vias and porous structures that penetrate the entire wall thickness can have additional advantages, including allowing droplets to be deposited from the opposite side of the wall in manufacturing, and allowing vias or pores to be backfilled with air to promote movement of the droplet toward and into the sample's fluid pathway, thus increasing the amount of detection reagent mixing with the sample. At least part of the fluid pathway may be a porous medium. By forming at least part of the fluid pathway from a porous medium, the capture components and/or detection reagents may be retained within the pores of the wall of the fluid pathway. The porous medium may be a porous wall, or a nanoporous membrane or a nanoporous block copolymer or a polymer foam or a metal foam.

In diffusion configurations, droplets containing detection reagent are ideally not carried away by sample flow if this would move them away from their corresponding capture components. This can be facilitated in several ways, each of which creates a “temporary matrix” that holds the droplet, in liquid form, until it is deployed. For example, the droplets can be of higher viscosity; be disposed in indentations; and/or a flow controller could reduce the detection reagent being carried away. High viscosity limits diffusion upon contact with the sample but increases mobility after dissolution. High viscosity can be achieved by adding trehalose, sucrose or glycerol, or any of the mentioned hygroscopic compounds that reduce water vapour pressure. As an extension of high viscosity, the droplets could contain a gel matrix or a matrix that dissolves/reacts with the sample. An example of a degradable matrix is a polysaccharide-based gel that degrades through action of the amylase enzyme in saliva, for assays where the sample is saliva. Alternatively, a cleavable crosslinked polymer adsorbed onto the surface of the droplet may be used. A further option would be a matrix in the droplet or a protective coating on the droplet which initially prevents the inner contents of the droplet from being carried with the flow, but then degrades (for example through a reaction or through dissolution) with time, thereby allowing the inner contents to diffuse to the region of capture components. The sample could also be doped with species that would promote such disintegration. In diffusion configurations, the maximum distance between disposed detection reagent and its corresponding capture component is given by the detection reagent's diffusion length x:

$$x \approx 2 \sqrt{Dt} \qquad ( 1 )$$

where t is the time allowed for the detection reagent to reach the corresponding capture component, and D is the diffusion coefficient of the detection reagent in its medium. Here, the medium can be the sample, the liquid of the droplet containing the detection reagent, or a mixture of the two. Equation 1 is plotted in FIG. 7 for IgG antibodies in water (D=4×10⁻¹¹ m²s⁻¹) and for IgG antibodies in mucus (D=3×10⁻¹¹ m²s⁻¹).

The assay cartridge may further comprise a flow controller configured to reduce the bulk movement of the sample in the vicinity of the capture components.

The flow controller may be required to slow the bulk movement of the sample sufficiently so that the detection reagents can bind to the target components and move to the capture components via diffusion. In diffusion configurations, a lower sample flow rate can also reduce the volume of droplets with detection reagent that is dragged away from the test site, if the viscosity of those droplets is higher than the viscosity of the sample.

The flow controller may effectively halt the bulk fluid flow. Alternatively, the bulk fluid flow may be reduced to 1 mm/minute, 0.5 mm/minute, 0.25 mm/minute or even substantially 0.0 mm/minute, i.e. stationary, so that the diffusion of the components within the sample is significant.

The flow controller may be provided distally of the capture components. By placing the flow controller distally, or downstream, of the capture components, the flow of sample into the fluid pathway is unimpeded thereby enabling the sample to be quickly introduced into the cartridge. The flow controller then acts to slow the flow of the sample once it has reached the capture components.

The flow controller can take any form that is effective in slowing the flow. The flow controller may be a capillary stop or a narrow or tortuous path. Alternatively, the flow controller may be provided by the geometry of the fluid pathway itself in the case where the fluid pathway is a well. The sidewall or sidewalls of the well provide the flow controller as they prevent the sample from flowing further and cause the sample to stop in the vicinity of the capture components that are applied to the base of the well or to the wall or walls near the base of the well.

In some embodiments, a porous-structure pump may be provided distally of the flow controller.

The assay cartridge may further comprise a physical barrier configured to divide the fluid pathway into a plurality of parallel flow channels.

Each flow channel may include a detection reagent and corresponding capture component. The flow channels may accommodate different detection reagents and their corresponding capture components. This configuration thereby enables a number of different detections reagents to be deployed for the same liquid sample and the same time without risk of cross-talk. The parallel flow channels may be provided within a single fluid pathway by the provision of a dividing wall running axially along the fluid pathway. The dividing wall may run the full length of the flow channel, or it may be discontinuous. The dividing wall may be the full height of the fluid pathway so that there is no fluid communication between the sub-flow-pathways at either side of the dividing wall. Alternatively, the dividing wall may provide an incomplete division of the fluid pathway. It may extend partially through the fluid pathway. Alternatively, the fluid pathway may divide to form two or more entirely independent fluid pathways.

The detection reagent and capture components may comprise antibodies. The detection antibodies may be fluorescently labelled or labelled with a scattering object.

The detection reagent and capture components may both comprise single-stranded oligo- or polynucleotides. Alternatively, the detection reagents and capture components may not be single-stranded, for example, they may be a g-quadruplex.

In addition to the capture components and detection reagents that are immobilised within the fluid pathway, other reagents may be incorporated into the fluid pathway and/or the sample collection unit to perform auxiliary functions within the cartridge. For example, labelling reagents may be present in the fluid pathway. Furthermore, other reagents that otherwise process the sample may be included in the fluid pathway.

The sample collection unit may be a porous structure which contains reagents. The porous structure may be a swab, sponge, nitrocellulose membrane, or one or more hydrophilic grooves or channels for saliva collection.

The porous structure may be pre-prepared to comprise reagents so that processing of the sample can be initialised.

The porous structure may be configured to indicate whether it is substantially saturated by the liquid sample. This may be achieved by way of a colour change.

The assay cartridge may further comprise a channel downstream of and/or distally positioned to the capture components that contains a confirmation element configured to show when the liquid sample is present in the channel. The confirmation element may comprise a transparent element with an angled surface, such that in absence of sample liquid it reflects the colour from a side wall, and in presence of the sample liquid it transmits a different colour from the bottom wall.

The assay cartridge may further comprise a detection reagent disposed with the capture components. The detection reagent may be a fluorescent or chemiluminescent molecule, enzyme and its substrate that produces a colorimetric signal.

The detection reagent and its corresponding capture component may target household proteins. In this context, a household protein is a protein for which concentration is relatively stable over time and similar for different sample sources, i.e. human, animal. This provides a benchmark or reference against which concentrations of other target components can be measured. This can enable the data obtained to be corrected to take into account variability of the total concentration of molecules in the sample. Alternatively or in addition, combination or marker proteins can be used as a stable reference if a weighted sum of their abundances is stable, that is, if their abundances typically counterbalance each other, for example albumin and hemopexin.

The assay cartridge may further comprise one or more target components immobilised within the fluid pathway. By immobilising one or more target components within one of the fluid pathways, the cartridge is provided with a reference that is indicative of flow behaviour. The results obtained from the other target components can be normalised using the data from the deposited target component.

The liquid droplet within which the detection reagents are provided may include a degradable shell. The degradable shell holds the liquid droplet thereby providing a physical barrier preventing the liquid droplet from being absorbed onto a substrate or wetting a surface or wicking away into a different part of the assay cartridge. It can also prevent the droplet from being dragged by the flow of liquid sample, or reduce the amount that is being dragged by the flow or reduce the distance by which the droplet gets dragged. The degradable shell also creates a microclimate inside the liquid droplet that can reduce evaporation and maintain a controlled chemical environment for the detection reagents.

Furthermore, according to the present invention there is provided an apparatus for detecting the presence and/or the amount of a target component in a sample of biological fluid, the apparatus comprising: an assay cartridge as heretofore described and a detector detecting the presence and/or the amount of the emitted light to provide an indication of the presence and/or the amount of the target component within the sample.

The apparatus can be encapsulated in a single housing including both the assay cartridge and the detector. Alternatively, the detector may be provided in a separate housing, sometimes referred to as a “reader”, that is distinct from the assay cartridge. The housing containing the detector may include a slot or opening sized and configured to accommodate the cartridge so that the cartridge can be introduced into the housing to enable access to the cartridge by the detector. The reader may also include other integers such as a light source and data collection, processing and storage capabilities.

The apparatus can detect one or more target components from a single sample. For example, two, three, four, five, ten, twenty or more separate targets components can be analyzed from a single sample of biological fluid. The analysis may be binary and merely indicate the presence or absence of a target component. Alternatively or additionally, the analysis may be quantitative and may give an indication of the amount of a target component present in the sample.

The apparatus may further comprise an excitation source configured to enable TIR (total internal reflection) illumination. Additionally or alternatively other emission-based optical assays may be deployed including fluorescence, phosphorescence, chemiluminescence, Raman, Rayleigh or Mie scattering, reflection, and absorption (including chromogenic mechanisms). Observations can be made in bright field and/or dark field mode.

The apparatus may further comprise a component for acoustic mixing. Acoustic mixing may be achieved via the provision of ultrasonic surface acoustic waves (SAW)) which will speed up the diffusion of the components within the sample and therefore shorten the time required to achieve binding and therefore detection of components within the sample liquid.

The component providing the acoustic mixing may be an actuator (e.g. piezoelectric) can be on chip or in the reader, or in a magazine feeding the reader or in an incubation device that is separate from the reader (touching the chip). The actuator must be in direct or indirect mechanical contact with at least one wall of at least one flow path or well. Between said wall(s) and said actuator, and impedance matching material may be present.

The apparatus may further comprise an optical readout component. The optical readout component may be a camera; specifically, it may be a CMOS or a CCD image sensor with one or more lenses or a CMOS or a CCD image sensor placed in close proximity to the test site so that no lenses are required.

The apparatus may further comprise an optical mask in the optical path between the bound detection reagent and the optical readout component. This applies to optical detection methods. The optical mask is configured such that light emitted by detection reagent away from the capture components is blocked thereby reducing background illumination from the detection reagents. The mask may be an opaque pattern on a surface of any optical component in the optical path, for example on the interior surface of a transparent wall at the test site of the device. This configuration enables low cost of fabrication as it may be produced by the same means as spotting of capture components and reagents. If the entire area around capture components is masked, the chip might not require (expensive) passivation steps to prevent nonspecific binding of detection reagent. Alternatively or additionally, the mask may be provided on the exterior surface of a transparent wall at the test site of the device.

The mask may be formed by a pattern in an opaque plate in the optical path. The mask may be made of elements that are switchable between an opaque and a transparent state, for example the elements may be pixels of an LCD screen.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

The invention will now be further and more particularly described, by way of example only, and with reference to the accompanying drawings, in which:

FIGS. 1 to 4 show, schematically, various permutations and combinations of detection reagents and capture components;

FIG. 5 shows an embodiment that allows a check that detection reagents have reached the capture component locations;

FIG. 6 shows a multi-lane configuration of the fluid pathway;

FIGS. 7 to 9 show possible detection reagent and capture component locations in embodiments including an elongate flow pathway;

FIGS. 10 to 13 each comprise a top view and a side view of embodiments of a cartridge showing the locations of the detection reagents in various indentations;

FIG. 14 shows a graph of calculated diffusion length vs. time for IgG antibodies in water and in mucus;

FIGS. 15 and 16 show further embodiments of the cartridge focusing on the introduction of the liquid sample into the assay cartridge;

FIG. 17 shows a TIRF-based CRP sandwich immunoassay where detection reagents and capture antibodies were disposed on opposing walls and where the spots of detection antibody were dry until introduction of the sample; and

FIG. 18 shows a TIRF-based PIGF sandwich immunoassay where detection and capture antibodies are both disposed on the TIRF-illuminated wall and where the spots of detection antibody were liquid until introduction of the sample.

### DETAILED DESCRIPTION

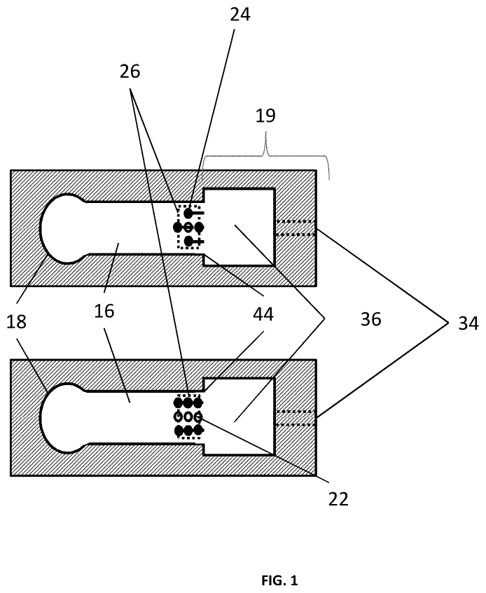

Referring to FIG. 1, there is provided an assay cartridge 10 for detecting a target component 20 in a liquid sample. The cartridge comprises a sample collection unit 12, in which the sample collection unit 12 has an inlet 18 which is configured to introduce the liquid sample 14 into the cartridge 10. The liquid sample 14 is able to flow through a fluid pathway 16 commencing at its proximal end at the sample collection unit 12 and extending distally through the cartridge 10 including one or more capture components 22 immobilised within the fluid pathway 16. Therefore, the terms proximal and distal are used to define positions relative to the sample collection unit 12 where the liquid sample 14 enters the cartridge 10. The fluid pathway 16 starts at the sample collection unit 12 and continues through the cartridge 10 until it reaches the capture components 22. The fluid pathway 16 then continues downstream of (in use), or distally from, the capture components 22. One or more detection reagents 24 provided proximally of or level with the capture components 22 each contained within a liquid droplet 15. The detection reagents 24 are level with the capture components 22 and therefore equidistant from the proximal end of the fluid pathway 16 so that the liquid sample 14 will come into contact with the detection reagents 24 at the same time as it comes into contact with the capture components 22 because they are the same distance from the inlet 18.

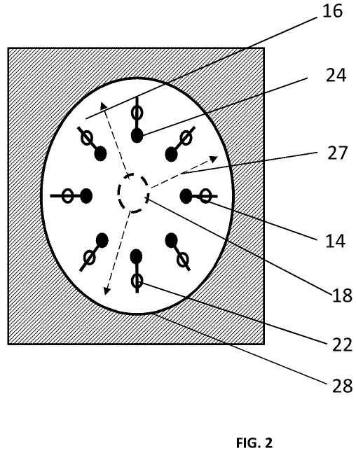

In the example shown in FIG. 1, the fluid pathway 16 is substantially linear as the fluid sample 14 flows from left to right as illustrated. The left hand end of the fluid pathway 16 is therefore the proximal end, owing to its location adjacent to the sample collection unit 12. In FIG. 2, the inlet is central and therefore the flow is radially outwards in all directions and therefore the proximal end of the flow pathway 16 is the centre of the well and the distal end of the flow pathway 16 is adjacent to the circumferential wall of the well.

As shown in FIGS. 1 and 2, the detection reagents 24 are in the form of liquid droplets which enables them to be combined with the sample such that they undergo bulk movement with the liquid sample 14. As the liquid sample 14 moves over the detection reagents 24, they are dragged along with the bulk movement of the liquid sample 14 and, because the flow of the liquid sample 14 is substantially laminar, they create streaks of detection reagent 24. The detection reagent 24 is configured to bind to the target components and move to the capture components 22 along the fluid pathway 16.

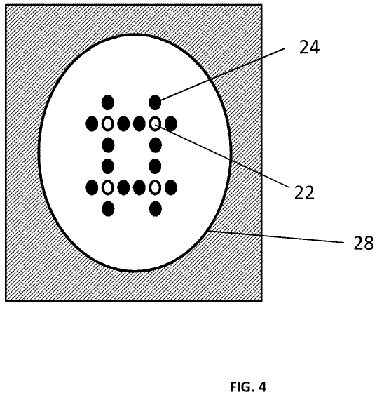

In FIG. 2, the detection reagents 24 are transported through bulk movement of the sample until they reach the vicinity of the detection reagents and the capture components, at which point the bulk flow is slowed sufficiently for diffusion to predominate. In FIGS. 1 and 4, the detection reagents 24 are transported predominantly through diffusion.

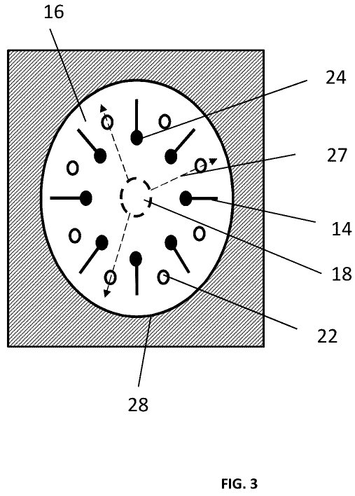

As shown in FIGS. 1 to 4, the detection reagent 24 is contained within a liquid droplet 15. The liquid droplets 15 in the illustrated embodiments have a circular cross section and are part-spherical. Alternatively, in embodiments not shown in the accompanying drawings, the liquid droplets can be non-spherical such as a rectangular liquid droplet. Liquid droplets that have a non-circular cross section can be created through manipulation of the surface tension of the liquid droplet 15 when it is deposited at its intended location on the fluid pathway 16. At these locations, or reagent sites, the surface of the fluid pathway is provided with a surface coating (not shown) to help manipulate or mould the liquid droplet 15 into a suitable shape. For example, a hydrophilic area may be provided which encourages the liquid droplet to overcome surface tension and spread across the surface. This hydrophilic area may then be bounded by a hydrophobic barrier which halts the spread of the droplet. The droplet will tend to assume the shape of the hydrophilic area in this example. This area may be rectangular, square or an irregular shape dictated by the geometry of the fluid pathway 16.

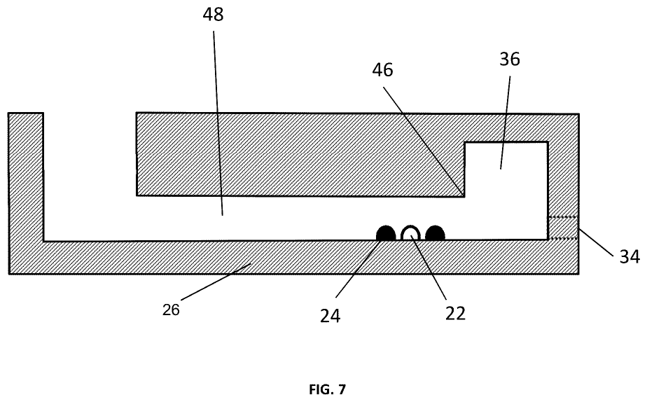

Referring to FIG. 7, the capture component 22 is deposited onto an optical element 26. The capture component 22 is deposited onto the optical element 26 via by printing. The optical element is a prism, dove or a cuboid optical element. The capture component 22 can be an antibody or a fragment thereof, a peptide, or a nucleic acid.

Referring to FIGS. 1 to 4, there is shown a flow controller 19 to help control fluid flow through the fluid pathway 16. The flow controller 19 can include one or more of the following: a capillary channel; a narrow, a long and/or a tortuous path; a capillary stop; a capillary stop with a vent or gas buffer or a flow resistor. As shown in FIG. 1, the fluid controller 19 may comprise, but is not limited to, a vent 34, a chamber 36 containing gas buffer, a flow resistor 38 or a capillary stop 44. In some examples, the capillary stop 44, the vent 34 and the chamber 36 containing gas buffer are used in combination to control the flow of the liquid sample through the fluid pathway 16. The flow controller 19 is provided for each fluid pathway 16. For example, the number of fluid controller 19 will be T₁, T₂, T₃ and corresponds to the number of N₁, N₂, N₃ fluid pathway 16 provided on the cartridge 10. The flow controller 19 is provided distally of the capture components 22. By placing the flow controller distally, or downstream, of the capture components 22, as shown in FIG. 1, the flow of sample into the fluid pathway 16 is relatively unimpeded thereby enabling the sample to be quickly introduced into the cartridge 10. The flow controller 19 then acts to slow the flow of the sample once it has reached the capture components 22. The flow controller 19 is taken to be any form that is effective in slowing the flow. The flow controller 19 is required to slow the bulk movement of the sample sufficiently so that the detection reagents 24 can bind to the target components and move to the capture components 22 via diffusion.

Referring to FIGS. 2 to 4, there is shown an alternative embodiment of a fluid pathway 16, in which the fluid pathway is a well 28. As shown in FIGS. 2 to 4, there is shown the co-location of the capture components 22 and detection reagents 24 within the well 28. The capture components 22 can be equal to or more than the number of the detection reagent 24 located within the well 28. In another example, the number of capture components 22 can be less than the number of detection reagents 24 located within the well 28. The inlet 18 for receiving the liquid sample is located at the center of the well, as shown in FIGS. 2 to 4, where the detection reagents 24 are positioned nearer to the inlet 18.

The flow controller 19 is provided by the geometry of the fluid pathway 16 in the case where the fluid pathway is a well 28, as shown in FIGS. 2 to 4. The sidewall or sidewalls of the well 28 provide the flow controller 19 as they prevent the sample from flowing further and cause the sample to stop in the vicinity of the capture components that are applied to the base of the well or to the wall or walls near the base of the well 28.

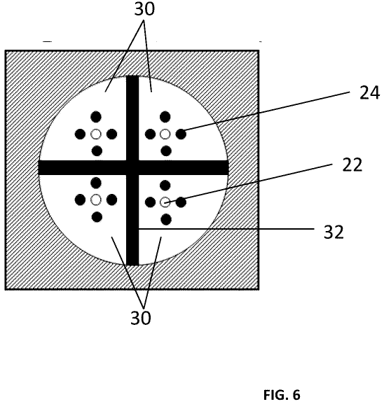

FIG. 6 shows multiple lanes with a physical barrier 32 provided between the lanes. Referring to FIG. 6, there is provided a cartridge 10 comprising a sample collection unit 12, an inlet 18 for receiving the liquid sample and multiple lanes 30. A physical barrier 32 such as a pair of walls is provided to separate the lanes as shown in FIG. 6. Without the walls 32, through diffusion, detection reagents 24 for one target component may cross-over to capture components for another target component; thus, cross-talk (unintended affinity) between the two might contribute to background signal and increase cross-talk between multiple assays on one device.

FIG. 6 shows an embodiment with multiple lanes 30, in which each of the lanes 30 can be used to test for a different capture component 22. Referring to FIG. 6, there is shown a physical barrier 32 i.e. a wall. The physical barrier 32 is configured to divide the fluid pathway 16 into a plurality of parallel flow lanes or channels 30. This configuration thereby enables a number of different detections reagents 24 to be deployed for the same liquid sample 14 and the same time without risk of cross-talk. One or more capture components 22 is deposited in each of the lanes 30 whereby the capture component is deposited onto the optical element 26. As the liquid sample 14 comes into contact with the detection reagents 24 and capture components, the bulk motion of the flow is slowed so that diffusion predominates and allows the target components to reach and react with the detection reagents and the capture components. A fluid controller 19 is provided downstream of the capture components 22 in order to control the flow of the liquid sample along each of the lanes 30 and to facilitate an initial, rapid fill of the fluid pathway and then a considerable slowing of the bulk flow to such a point where diffusion can predominate.

The physical barrier 32 i.e. the walls separating the well 28 into four quarters extend over the entire fluid pathway 16 from the sample collection unit 12 to the well base. However, for TIR methods in particular, such a separating wall 32 can cause unwanted reflections and scattering of the excitation and emission light.

In an alternative embodiment, there is provided a partial barrier or a soft texture not shown in the accompanied drawings that runs along the fluid pathway 16. The physical barrier 32 would be at a partial height and would not extend all the way to the top of the sample collection unit 12 but to provide a gap. This ensures that the flow channels or lanes 30 are partially separated. In one example, the partial barrier or the soft texture is a semi-permeable membrane that is designed to reduce cross talk between multiple parallel flow channels or lanes 30 without contacting the optical surface and thereby risking interference with the TIR measurements.

Channels are typically of rectangular cross-section due to two-dimensional nature of current mass fabrication processes. In many microfluidic devices, channel dimensions are limited by the amount of available sample and/or cost of reagent. However, in TIRF-based detection systems, channel height is less of an issue. For the case where reagents are disposed on the wall along which the TIRF evanescence is generated, as long as Reynolds numbers remain sufficiently low (<1e3), laminar flow will keep reagent concentrated near the wall. The fluid dynamics of the illustrated embodiments are such that the fluids all execute laminar flow throughout. Turbulence is minimized so that the dominant lateral motion arises from diffusion, not turbulence.

In some embodiments, down-stream passive pumping structures may be provided that create a second flow regime that is slower than the initial sample fill up to the test site The flow rate created by the pumping structures must be slow enough for target components to reach the capture components by diffusion, specifically, flow velocity over the test site must be less than 10 mm/min, less than 5 mm/minute, less than 2 mm/minute or even less than 1 mm/minute.

Besides capillary driven flow, low flow rates may also be achieved using evaporation. The chamber containing the gas buffer 36 after a capillary stop 44, may contain e.g. dry air or dry nitrogen gas (which requires device packaging to be sealed until use). Its initial humidity and volume may be designed such that humidity remains sufficiently low so that the evaporation rate does not drop significantly for the required duration of the assay. Alternatively, it may be designed such that it saturates with water vapor during the assay measurement, thus stopping the flow and preventing the test site from drying out.

In some embodiments, not illustrated, a vent 34 may be provided after the capillary stop 44. This demands less real estate on the cartridge, but it leads to a variable flow rate that is dependent on ambient humidity thus limiting the operating conditions.

In absence of second (slow) flow regime, a capillary stop 44 is required downstream of the test site. It must be no further from the capture components 22 in the test site than the length of streaks of detection reagent (for upstream or proximal deposition in channel geometries). For diffusion-based assays (co-located capture components and detection reagents), the capillary stop 44 must be as close as possible to the capture components 22 but far enough to not interfere with the assay (e.g. for optical detection, the meniscus may need to be outside the field of view of the detection element in order to avoid intense background light from reflection off the meniscus).

The combination of capillary stop 44 with evaporation into a chamber 36 containing the gas buffer or the vent 34 can also be used to concentrate the sample including capture components and detection reagents at the test site. This can be effective when flow velocity due to evaporation is higher than the velocity of diffusion of target components or detection reagents. Evaporation rate is determined by meniscus area and curvature and by humidity; diffusion distance vs. time x(t) is determined by diffusion constant D according to x≈2\sqrt{Dt}

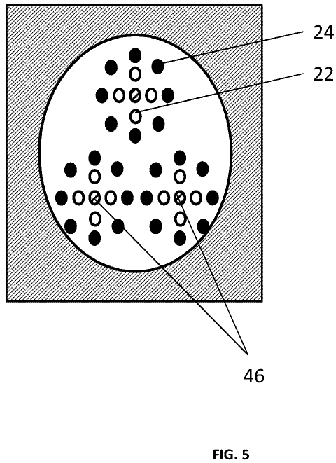

Referring FIG. 5, there is provided a check that detection reagents have reached the capture component locations. FIG. 5 shows an example of co-located capture component 22 (“Ab1”) and detection reagent (“Ab2”) 24 within a well 28. The central spot 46 is a spot of capture components 22 that are complementary to the detection reagents (“anti-Ab2”) 24.

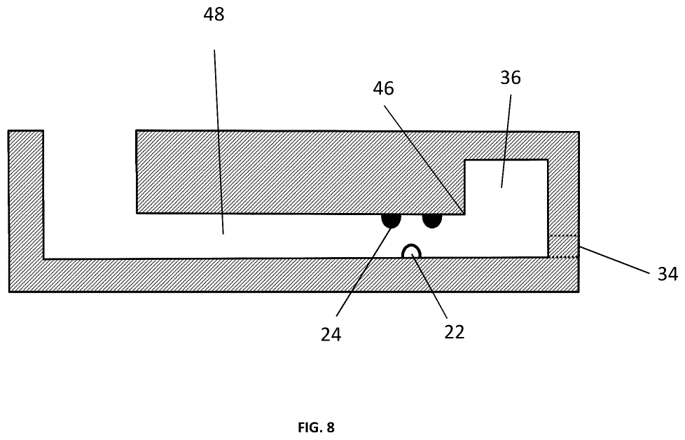

Referring to FIGS. 7 to 9, there is shown possible detection reagent 24 and the capture component 22 locations in embodiments including an elongate flow pathway 48. There is also provided a capillary stop 44, a chamber 36 containing gas buffer and a vent 34 to control fluid flow, as shown in FIGS. 7 to 9.

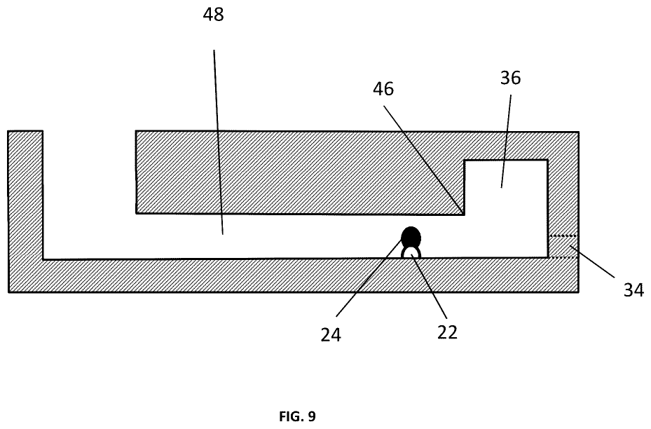

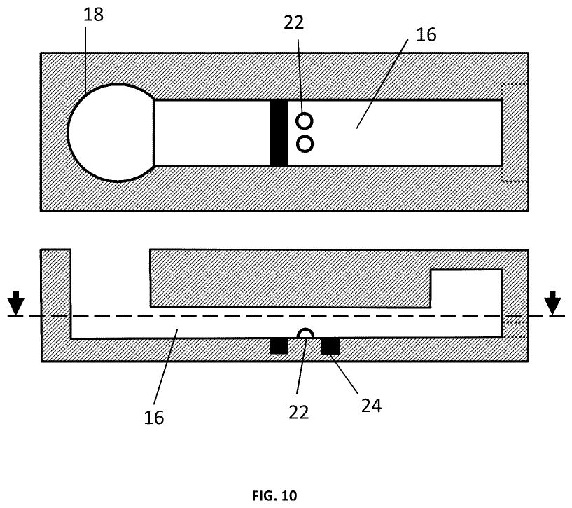

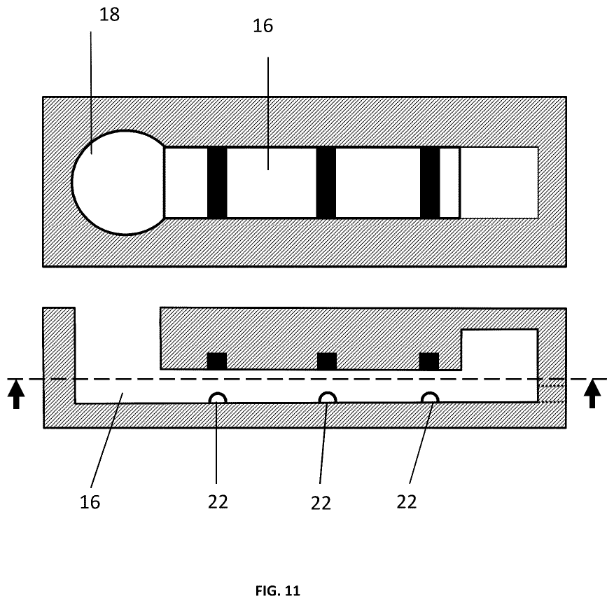

In some examples, illustrated in FIGS. 10 to 13, a surface of the fluid pathway is provided with one or more indentation, such as a recess, a trough, a ditch, a trench, a groove, a gully, a via that is essentially perpendicular to the surface, or a porous structure in order to provide a location for the deposition of the liquid droplets. This can have several advantages, including precise location of the deposited droplets, reducing evaporation by reducing surface-to-volume ratio, having the option to deposit droplets from the opposite side of the pathway's wall (in the case of a via or porous structure), increasing the amount of detection reagent per unit length of the fluid pathway, and reducing the amount of detection reagent that is dragged by the sample flow. The latter advantage applies to assays in diffusion configuration. As an example only, the indentation is provided where the detection reagents 24 are positioned within fluid pathway 16. The liquid droplet is at a suitable configuration to enable it to position itself within the indentation on the surface of the fluid pathway. FIGS. 9 to 13 also show that the fluid pathway 16 includes the location of the capture component 22 or components and any intervening geometry. In some examples, an indentation on the surface of the optical element may be suitable to provide a location for deposition of the capture component. In some embodiments, where indentation is unsuitable on an optical element, a gasket can be provided between the optical element and the fluid pathway to help position the capture component.

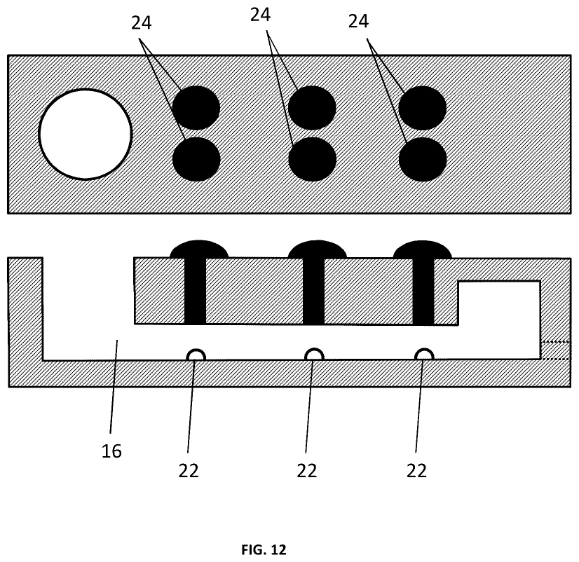

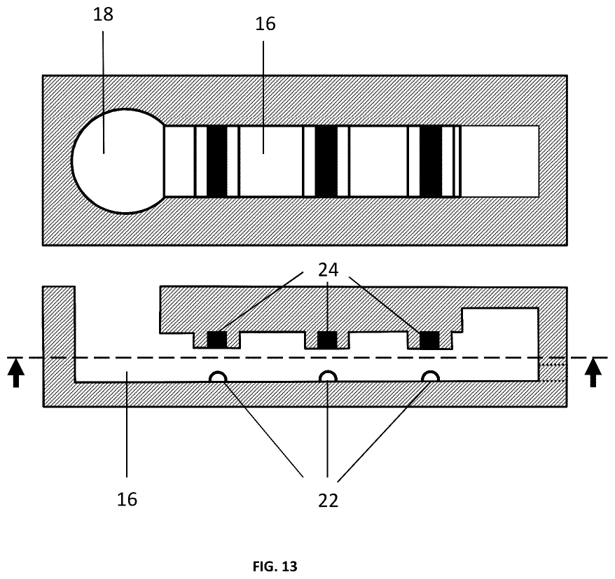

FIG. 13 shows a configuration of the fluid pathway 16 that is particularly suited to liquid droplets of detection reagent. The fluid pathway 16 is provided with a series of indentations which are filled with the detection reagent in liquid form. The indentations in the fluid pathway 16 protect the liquid droplets of detection reagent during the initial, rapid, liquid fill so that they retain their position for the diffusion phase. Although in the illustrated embodiment, each of the detection reagents is housed in an indentation, other modifications to the fluid pathway 16 geometry may be selected to protect the liquid detection reagents. For example, a bump or protrusion may be provided at the upstream side of the detection reagent and this will provide a similar sheltering effect for the liquid detection reagent.

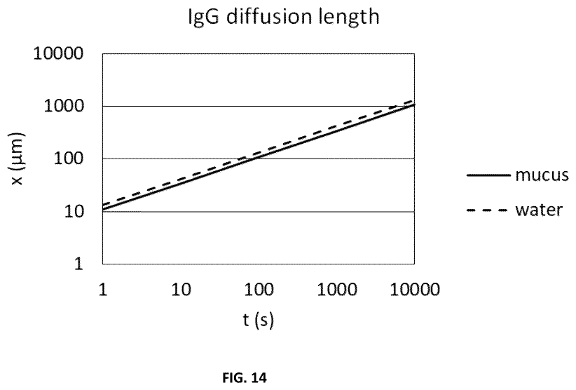

FIG. 14 shows a graph of calculated diffusion length vs. time for IgG antibodies in water and in mucus; diffusion length is an estimate for the distance that a molecule is able to travel when driven by Brownian motion. The calculations are based on measured diffusion constants as described above.

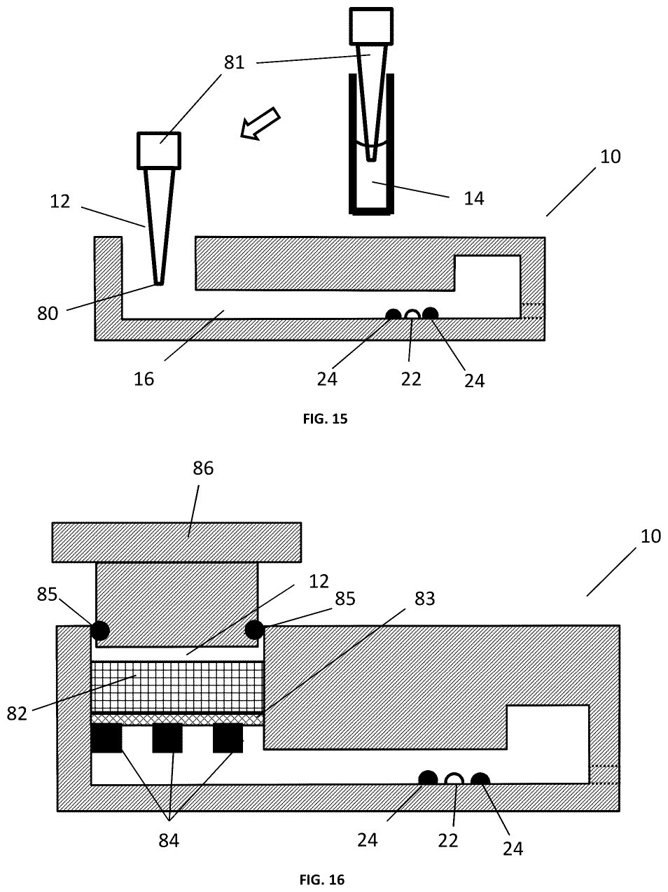

FIG. 15 shows an assay cartridge 10 in which the sample collection unit 12 is an opening 80 at the proximal end of the fluid pathway 16. The opening is provided in the upper surface of the cartridge 10 so that the introduction of the fluid sample 14 is aided by gravity. The liquid sample 14 is introduced into the cartridge 10 using a pipette 81. The opening that provides the sample collection unit 12 is sized to accommodate the tip of the pipette 81 so that the fluid sample 14 can be introduced into the cartridge 10 accurately.

FIG. 16 shows an assay cartridge 10 in which the sample collection unit 12 comprises a pad of porous material 82 such as a sponge, a filter 83 and a support mesh 84. The pad 82 soaks up the fluid sample 14 as it is introduced into the cartridge 10. The structure of the pad 82 holds the fluid sample 14 in place and prevents it from easily leaving the cartridge 10 once it has been introduced to the cartridge. When sufficient fluid sample 14 has been collected, the cartridge 10 is closed using a plunger 86 which forms a seal around the opening in the cartridge 10 using an O-ring 85. The plunger 86 compresses the pad 82 and forces the fluid sample 14 through the filter 83 and into the fluid pathway 16. The support mesh 84 is provided to hold the filter 83 in place and to prevent it from moving into the fluid pathway 16. The filter 83 is provided to remove particulate matter and/or biological matter such as mucins from the fluid sample 14 that is undesirable and could interfere with functioning of the assay cartridge 10.

Within the context of this invention, a capture component and a detection reagent can be a protein or peptide, including an antibody or enzyme; an oligo- or polynucleotide, such as DNA or RNA; a modified oligo- or polynucleotide, such as a locked nucleic acid (LNA); an aptamer; a morpholino; a small molecule that may be grafted via a spacer molecule; a cell; a cell membrane; a membrane receptor; a viral particle; a glycan; a solid particle or bead coated with a reagent or other type of molecule or material that can have a ligand receptor type of interaction with the target component of interest. For optical detection, detection reagents may be labelled with a luminophore such as a fluorophore or a phosphor or a chemiluminescent molecule, or an enzyme and its substrate that produces a colorimetric or luminescent signal.

The detection reagent can also be any reagent including a cofactor or any molecule used to process the sample (e.g. sodium dodecyl sulfate used for lysing cells).

### Example 1

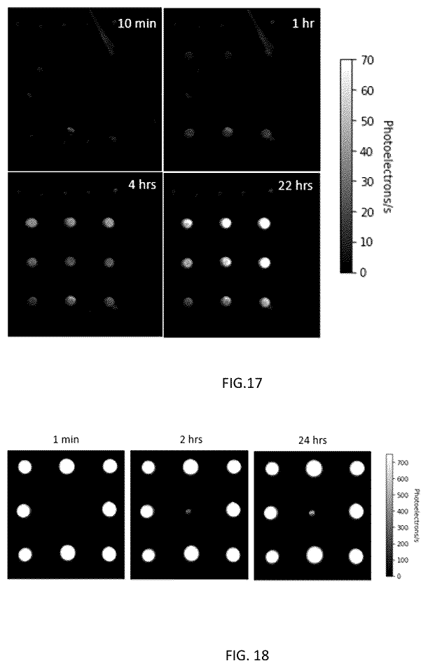

FIG. 17 shows a TIRF-based CRP sandwich immunoassay where detection and capture antibodies were disposed on opposing walls and where the spots of detection antibody were dry until introduction of the sample. Shown are four incubation times after filling a channel with a saliva sample from a healthy subject. The saliva was pre-filtered (5-μm pore size). Detection reagent (Alexa-647-labelled monoclonal IgG targeting native human CRP) in SSC buffer was contact-printed onto the top surface of a channel of cross-section 10×0.09 mm, in two lines spaced 3 mm apart with a spot pitch of 0.6 mm within each line. The capture component (monoclonal IgG targeting native human CRP) was deposited in print buffer as well (3 rows with 1-mm pitch on the bottom surface of the channel), but rinsed with MilliQ water prior to chip assembly. During imaging, the bottom surface was illuminated with TIRF.

### Example 2

FIG. 18 shows a TIRF-based human epithelial growth factor sandwich immunoassay where detection and capture antibodies are both disposed on the TIRF-illuminated wall and where the spots of detection antibody were liquid until introduction of the sample. Shown are three different incubation times after filling a channel with a solution of recombinant human epithelial growth factor in PBSA (3×10\hat{ }9 P1GF/μL and 4% BSA in phosphate buffer). Detection reagent (Alexa-647-labelled monoclonal IgG targeting human epithelial growth factor) in print buffer was contact-printed onto the bottom surface of a well of diameter 9 mm and depth of 0.6 mm, in a 2×2-mm square pattern. The capture component (monoclonal IgG targeting human epithelial growth factor) was deposited in print buffer as well, as a single droplet, but rinsed with MilliQ water prior to chip assembly.

Within the context of this invention, a target component can be a protein or peptide, including an antibody or enzyme or membrane receptor; an oligo- or polynucleotide, such as DNA or RNA; a cell; a small molecule; a viral particle; a glycan; a drug candidate, or other type of molecule or particle of interest.

By the term ‘liquid droplet’ used herein we mean a spot on the device comprising at least some liquid component e.g. that carries a reagent directly solubilised or suspended within it. For example, this includes liquids, gels, suspensions, or combinations thereof. The droplet may also include a degradable shell that releases its contents due to contact with the sample. A liquid can be a solution that includes a polymeric compound or compounds. The droplet may be a partial sphere formed when a liquid mass is deposited on to a surface. However, it should be understood that the term “droplet” also covers other shapes of fluid amalgam. For example, if the surface onto which the liquid is deposited is treated with one or more of a hydrophilic or hydrophobic layer, this may overcome the surface tension of the liquid and cause it to flow such that it has a non-circular footprint. Alternatively, adjacently placed and connecting liquid masses deposited in a pattern can maintain the pattern through contact line pinning. The footprint of the liquid droplet may be therefore, in addition to a circular footprint, rectangular, square or elliptical. It may even have an irregular shape which may be at least partially dictated by the packaging requirements of the fluid pathway. Within the context of this invention a liquid droplet ceases to exist when it is absorbed into a porous matrix, such as a nitrocellulose matrix or the liquid evaporates leaving behind a spot of dried matter that is no longer in solution.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Throughout the specification, unless context dictates to the contrary, the singular should be understood to encompass the plural. That is, “one” and “a” and “the” should be understood to encompass “at least one” or “one or more.”

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1. A chip-based optical detection system for detecting a target component in a liquid sample, the system comprising:
a chip comprising an interior volume;
a fluid pathway disposed in the interior volume of the chip;
an inlet fluidically connecting an external surface of the chip to the fluid pathway;
an optical element at least partially defining the fluid pathway; and
a flow controller configured to control a flow of the liquid sample through the fluid pathway,
wherein one or more capture components are immobilised on the optical element,
wherein one or more detection reagents are provided in a proximity of the capture components, and
wherein the flow controller is configured to control the flow of the liquid sample along the fluid pathway such that the detection reagents are brought into contact with the capture components via diffusion.

2. The system according to claim 1, wherein the one or more detection reagents and/or the one or more capture components are contained within a liquid droplet.

3. The system according to claim 2, wherein at least one of the liquid droplets comprises an additive that minimizes evaporation.

4. The system according to claim 1, wherein the fluid pathway includes one or more indentations.

5. The system according to claim 1, wherein the optical element comprises one or more indentations.

6. The system according to claim 1, further comprising a porous-structure pump provided distally of the flow controller.

7. The system according to claim 1, further comprising a channel downstream of the capture components that contains a confirmation element configured to show when the liquid sample is present in the channel.

8. The system according to claim 2, in which the liquid droplet within which the detection reagents are provided includes a degradable shell.

9. An apparatus for detecting the presence and/or the amount of a target component in a sample of biological fluid, the apparatus comprising: the system according to claim 1 and a detector configured to detect the presence and/or the amount of the emitted light and to provide an indication of the presence and/or the amount of the target component within the sample.

10. The apparatus according to claim 9, wherein the apparatus further comprises an excitation source configured to enable TIR 43 illumination.

11. The apparatus according to claim 9, further comprising a component for acoustic mixing.

12. The apparatus according to claim 9, further comprising an optical readout component.

13. The apparatus according to claim 12, further comprising an optical mask in the optical path between the bound detection reagent and the optical readout component.
